(19) United States
(12) Patent Application Publication
(10) Pub. No.: US 2005/0209567 A1
(43) Pub. Date: Sep. 22, 2005

(54) Stress-reducing medical devices

(76) Inventor: Wilmer L. Sibbitt JR., Albuquerque, NM (US)

Correspondence Address: ROBERTS ABOKHAIR & MARDULA, SUITE 1000, 11800 SUNRISE VALLEY DRIVE, RESTON, VA, 20191, US

(21) Appl. No.: 11/142,220

(22) Filed: May 31, 2005

Related U.S. Application Data
(63) Continuation-in-part of application No. 10/693,082, filed on Oct. 27, 2003.
(60) Provisional application No. 60/578,995, filed on Jun. 12, 2004.

(51) Int. Cl. A61M 5/00

(52) U.S. Cl. 604/187; 604/264; 606/170; 604/80; 128/898; 128/206.24

(57) ABSTRACT

Medical devices and consumables have been altered by changes on their surfaces or components consisting of simple or complex color patterns, visual designs, drawings, art work, embedded designs, copyrighted images or figures, changes in the shape of the medical device in an artistic, abstract, or visual manner, and other visual and physical modifications intended to improve the physical and psychological environment of the patient and the health care worker, and to be used as well to visually identify a particular medical device, component, solution, or medication.

### CROSS REFERENCE TO RELATED APPLICATIONS

This application is a continuation-in-part of application Ser. No. 10/693,082, filed Oct. 27, 2003. The Ser. No. 10/693,082 application is incorporated by reference herein, in its entirety, for all purposes. This application also claims priority benefit under 35 U.S.C. § 119(e) of provisional application No. 60/578,995, filed Jun. 12, 2004. The 60/578,995 application is incorporated by reference herein, in its entirety, for all purposes.

### FIELD OF THE INVENTION

The present invention relates generally to the field of medical devices. More specifically, the present invention relates to the placement of images onto medical devices in such a manner as to provide a positive affect in and effect on patients and others who see the devices.

### BACKGROUND OF THE INVENTION

Although non-medical consumer goods typically may have many decorative aspects to make them more attractive to consumers, medical devices are typically bland in appearance and are devoid of intrinsic ornamentation. There is a long-standing tradition in medicine, from the manufacturer to the medical provider, that medical devices should be metallic or of a single color and devoid of decorations or ornamentation to give the appearance of sterility and to enhance the appearance of functionality. An exception to this is the name, identification numbers, information, and logo of the manufacturer, which are usually monochrome and imprinted, etched, or otherwise fixed to the surface. The logo is generally placed on one spot on the medical device or in a repetitive pattern. Certain texturing of monochrome surfaces, usually for enhancing grip on the device, can also be present, but these are for the user. These embellishments on medical devices are placed for regulatory, promotional, and instructional purposes for the health provider or user alone. These are not placed on the device for a separate therapeutic effect of benefit to the patient.

Some instances exist of ornamentation in medicine: small bandages, medical tape, stickers places over bandages, nursing and technician scrub suits, and clinic wear. These are not medical devices used in procedures—rather these are comfort measures that are applied after a procedure or to enhance the general care environment. The parent application Ser. No. 10/693,082 discloses the use of ornamental changes to butterfly needles.

Gloves have been disclosed with imprinted images. For additional details, refer to U.S. Pat. No. 5,357,636 to Dresdner, Jr. et al. A recapping device has been disclosed with imprinted images. For additional details, refer U.S. Pat. No. 5,209,738 to Bruno. A syringe filling mechanism has been disclosed having a part imprinted wit images. For additional details, refer to U.S. Pat. No. 5,220,948 to Haber et al. A testing device has been disclosed as having color coding and imprinted images. For additional details, refer to U.S. Pat. No. 5,099,857 to Baldo et al. A sharps container has been disclosed with imprinted images. For additional details, refer patent U.S. Pat. No. 4,955,477 to Bruno.

The emotional response of a patient to a medical device is an important issue in medicine that has, to date, been inadequately addressed. Patient dislike, fear, and anxiety of medical devices used in procedures (including needles, syringes, scalpels, gloves, etc) are a major problem in medicine and can interfere significantly with necessary medical care. This aversion to medical devices, which is common to both children and adults, is broadly termed “needle-phobia”, and can cause great emotional suffering in patients. However, for the great majority of medical devices, the monotony of the color and design of medical devices do not distract people from their fear, but rather intensify needle-phobia in patients, both adult and children. These cold and unattractive conventional medical devices are also not stimulating to physicians. Because medical devices generally appear monotone without bright colors, variegated patterns, or realistic or abstract design, they appear cold, hard, and frightening and enhance the sensation of pain and cause further fear, anxiety, and alienation of the patient. These devices are so monotonous and boring, the patient can focus only on the pain and harsh reality of their situation, while the physician, nurse, or technician is also bored by the monotony of the devices, and thus, is less alert, more alienated, and more likely to make a mistake or lose the monochrome device in the surgical field.

What is needed are medical devices that prevent and treat needle-phobia by incorporating bright designs, artistic alterations, realistic and abstract designs, and multiple colors that stimulate the patient's brain in a beneficial manner, provide a therapeutic effect, lessen patient suffering, and improve patient outcome.

A particular problem in medical care is discerning which tubing or catheters, particularly intravenous tubing or other catheter or medical device that goes from a particular source (usually a bottle or bag) into a destination (a pump, catheter or other delivery device, and ultimately, into the patient or instrument). If there are two or more of these tubes, confusion can result, causing misadministration of a drug or therapy.

Thus, what is also needed is a tubing scheme that permits multiple tubes, catheters, or other medical devices to be differentiated from one another to help ensure that two or more components be correctly assembled.

### SUMMARY OF THE INVENTION

One aspect of the present invention is that it provides enhanced medical devices and consumables with a predictable therapeutic effect for the health care providers and patients. These medical devices and consumables actually reduce fear of medical devices, improve the medical environment, reduce patient suffering, reduce medical and pharmacy errors, enhance patient care and outcome, and improve the emotional state of both patients and health care providers.

This invention includes medical devices and consumables with aesthetic modifications that provide a measurable functionality and therapeutic effect. These modifications consist of specific and general color patterns and graphic designs to attract and distract the patient's attention, reduce patient suffering, to have a predictable therapeutic effect, and to improve the general health care environment. These modifications are especially useful in children, but also beneficially affect adult patients and interest health care providers.

Use of these interesting and aesthetically pleasing devices and consumables actually prevent and treat needle-phobia (fear of medical devices) and make the bad experience of a medical procedure or therapy better, gentler, kinder, more interesting, and more meaningful. These devices also reduce medical, nursing, and pharmacy errors. The present invention provides colors, designs, graphic representations, and various coding schema for medical devices and consumables to be more interesting, stimulating, and more useful, and to provide a predictable and beneficial therapeutic effect on the patient.

Another aspect of the present invention is visual and/or textural coding of tubes so as to enable multiple tubes, catheters, or other medical devices to be differentiated from one another and thus, promote correct assembly of two or more components.

Upon further study of the specific descriptions and appended claims of this invention, further advantages and objects of this invention will become apparent to those skilled in the art.

### BRIEF DESCRIPTION OF THE DRAWINGS

FIG. 1 illustrates a syringe according to the prior art without an image imprint.

FIG. 2 illustrates a syringe imprinted with a repetitive colored geometric pattern.

FIG. 3 illustrates a syringe imprinted with an alternative repetitive colored geometric pattern.

FIG. 4 illustrates a syringe imprinted with a repetitive star pattern.

FIG. 5 illustrates a syringe imprinted with a repetitive star pattern with and contrasting colors on the flanges and the thumb rest.

FIG. 6 illustrates a syringe imprinted with a repetitive pattern based on a literary character motif.

FIG. 7 illustrates a syringe imprinted with an abstract pattern of swirls and stripes.

FIG. 8 illustrates a syringe imprinted with an abstract pattern of stripes.

FIG. 9 illustrates a syringe imprinted with an abstract pattern of polka dots.

FIG. 10 illustrates a syringe imprinted with an abstract pattern of non-colored outlines.

FIG. 11 illustrates a scalpel according to the prior art without an image imprint.

FIG. 12 illustrates a scalpel imprinted with a repetitive colored geometric pattern.

FIG. 13 illustrates a scalpel imprinted with an alternative repetitive colored geometric pattern.

FIG. 14 illustrates a scalpel imprinted with a repetitive star pattern.

FIG. 15 illustrates a scalpel imprinted with a repetitive star pattern with and contrasting colors on the flanges and the thumb rest.

FIG. 16 illustrates a scalpel imprinted with a repetitive pattern based on a literary character motif.

FIG. 17 illustrates a scalpel imprinted with an abstract pattern of swirls and stripes.

FIG. 18 illustrates a scalpel imprinted with an abstract pattern of stripes.

FIG. 19 illustrates a scalpel imprinted with an abstract pattern of polka dots.

FIG. 20 illustrates a scalpel imprinted with an abstract pattern of non-colored outlines.

FIG. 21 illustrates an intravenous fluid bag imprinted with a repetitive pattern based on a literary character motif.

FIG. 22 illustrates a needle with cap or shield imprinted with a repetitive pattern based on a literary character motif

FIG. 23 illustrates an introducer having a handle that is imprinted with a repetitive pattern based on a literary character motif.

FIG. 24 illustrates a skin biopsy device having a handle that is imprinted with a repetitive pattern based on a literary character motif.

FIG. 25 illustrates a medical glove imprinted with a repetitive pattern based on a literary character motif.

FIG. 26 illustrates a blood drawing device imprinted with a repetitive pattern based on a literary character motif.

FIG. 27 illustrates medical tubing according to the prior art without an image imprint.

FIG. 28 illustrates medical tubing imprinted with a coded coloration across substantially the entire tube surface.

FIG. 29 illustrates medical tubing imprinted with a coded coloration across only a striped portion of the tube surface.

FIG. 30 illustrates medical tubing imprinted with a coded coloration in a linear pattern.

FIG. 31 illustrates medical tubing imprinted with a coded coloration in a spiral pattern.

FIG. 32 illustrates medical tubing imprinted with a repetitive pattern based on a literary character motif

FIG. 33 illustrates medical tubing imprinted with a coded coloration in an alternative spiral pattern.

FIG. 34 illustrates medical tubing imprinted with coded symbols.

FIG. 35 illustrates medical tubing imprinted with a combination of coded coloration and coded symbols.

FIG. 36 illustrates medical tubing with a modified surface texture.

### DETAILED DESCRIPTION OF THE INVENTION

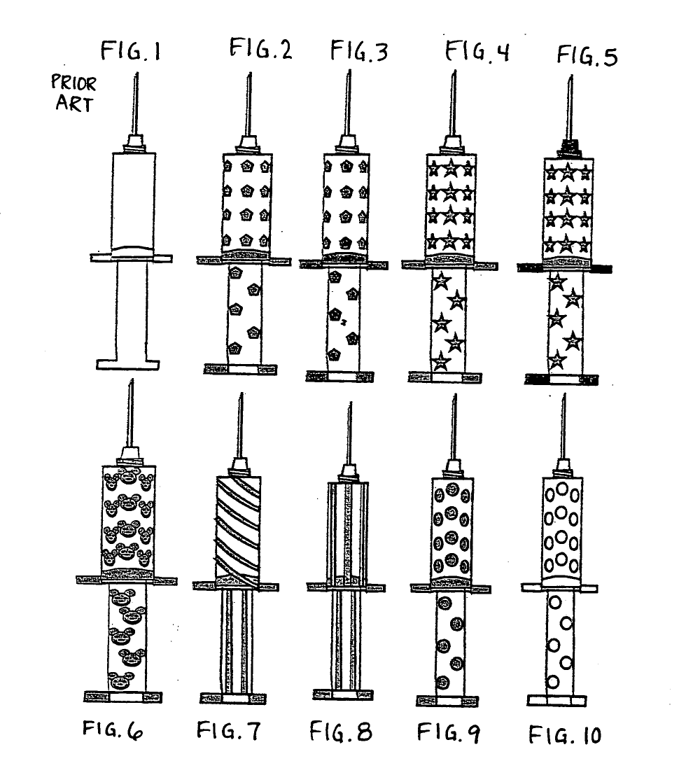

Referring to FIG. 1, a syringe according to the prior art without an image imprint is illustrated. This is an example of the existing medical devices and consumables formed of plastic, paper, metal, or other material and having a monotonous appearance, typically of a darker hue. This tends to make the medical device or consumable appear cold, hard, and menacing.

The present invention diminishes this negative design effect of prior art medical devices by dispensing with monotones, and making the surface of the medical device or consumable interesting and attractive. The present invention may advantageously be practiced by adding bright colors, hues, reflecting surfaces, translucency, patterns, and designs to the medical devices and consumables to make them interesting and distracting to the patient and to exert the predictable therapeutic effect.

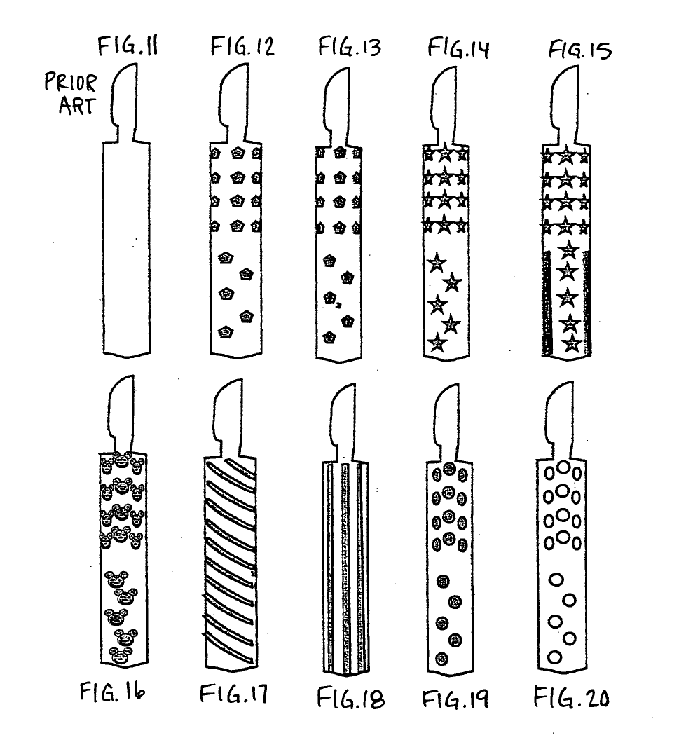

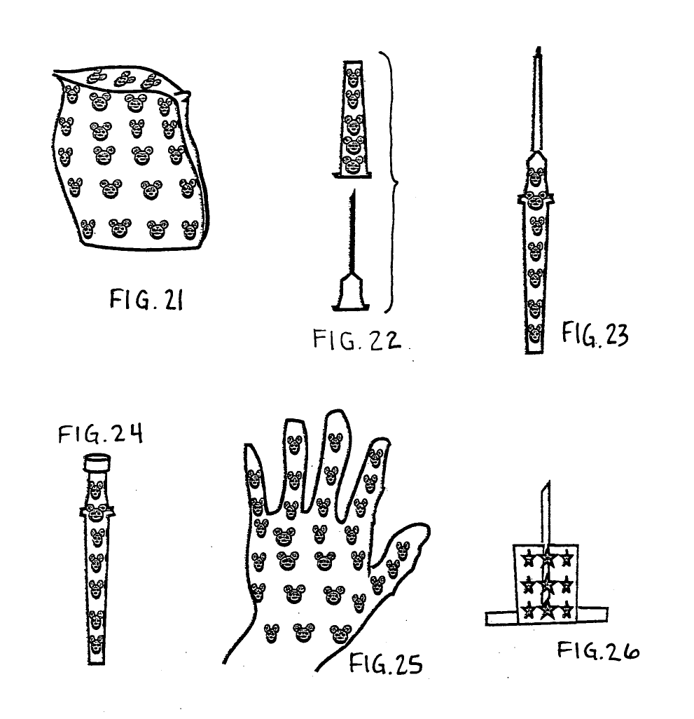

Referring to FIGS. 2-10, syringes are illustrated having imprints according to various exemplary implementations of the present invention. FIGS. 2-5 illustrate the use of repetitive colored patterns of various shapes on a syringe. Other geometric shapes may be used, the ones shown being but illustrative examples. FIG. 6 illustrates an example of a literary character (in this case Mickey Mouse) imprinted in a repetitive pattern on a syringe. Other literary characters may be used, the one shown being but an illustrative example. FIGS. 7-9 illustrate the use swirls, stripes, and polka dots as examples of abstract patterns on a syringe. Other abstract patterns may be used, the ones shown being but illustrative examples. FIG. 10 shows that the decoration of the syringe may be implemented with outlines of shapes in contrast to the shapes being filled in with color.

The images may be imprinted on both the barrel and the plunger portions of the syringe, or on either of the barrel or the plunger. The decorated plunger is of particular value, as the decorations can be seen through the barrel, and visualization of the contents of the syringe and the markings on the barrel remain unaltered, which is of benefit to the health care provider who may use a syringe.

Referring to FIG. 11, a scalpel according to the prior art without an image imprint is illustrated. This is another example of the existing medical devices and consumables that are formed so as to have an appearance that tends to make the medical device or consumable appear cold, hard, and menacing.

Referring to FIGS. 12-20, scalpels are illustrated having imprints according to various exemplary implementations of the present invention. FIGS. 12-15 illustrate the use of repetitive colored patterns of various shapes on a scalpel handle. Other geometric shapes may be used, the ones shown being but illustrative examples. FIG. 16 illustrates an example of a literary character (in this case Mickey Mouse) imprinted in a repetitive pattern on a scalpel handle. Other literary characters may be used, the one shown being but an illustrative example. FIGS. 17-19 illustrate the use swirls, stripes, and polka dots as examples of abstract patterns on a scalpel handle. Other abstract patterns may be used, the ones shown being but illustrative examples. FIG. 20 shows that the decoration of the scalpel handle may be implemented with outlines of shapes in contrast to the shapes being filled in with color.

These concepts, of bright, monochrome or non-monochrome, or surface designs are not limited to syringes (as shown in FIGS. 2-10) and scalpels (as shown in FIGS. 12-20) but to all catheters and medical devices and consumables, whether plastic, paper, metal, or other material with relatively flat or curvilinear or other contiguous surfaces where such shapes and surface colors and designs could be implemented, and these are also claimed. FIGS. 21-26 shows a number of other medical devices that could have these concepts implemented.

Referring to FIGS. 21-26, examples of other designer medical devices are illustrated. Referring to FIG. 21, an intravenous fluid bag imprinted with a repetitive pattern based on a literary character motif is illustrated. In similar fashion such images may be imprinted on bottles. Referring to FIGS. 22-24, a needle with a cap or shield, an introducer handle, and a skin biopsy device handle are illustrated as being imprinted with a repetitive pattern based on a literary character motif. Such imprinting may be used on other procedure devices, the ones shown being but illustrative examples. Referring to FIG. 25, a medical glove imprinted with a repetitive pattern based on a literary character motif is illustrated. In similar fashion such images may be imprinted on a gown, a mask, a head covering, or drapes. Referring to FIG. 26, a blood drawing device (for example, a Vacutainer) imprinted with a repetitive pattern based on a literary character motif is illustrated.

These are examples of patterns, both natural and man-made, that dramatically change the appearance of the medical device or consumable and make it more interesting and more distracting. Although the illustrated examples are portrayed in black and white, it is desirable that these images have variable bright colors, metallic and reflective surfaces, glittery surfaces, transparent, or translucent. Dramatic surface design images are also desirable. The images may mimic real physical forms, may portray fantasy images, or may be completely abstract.

These changes in color, design, and pattern could be integrated into the plastic or composition of the device or could be painted, printed, extruded, pasted, bonded, or otherwise fixed onto the surface of the medical device or consumable. This also includes the use of stickers bearing a design on an adhesive backing, which then placed onto the medical device. Designs including but not limited to a geometrical, repetitive, single, letters, numbers, or other design, fish, dolphin, whale or other sea-going or water-going animal, flying creatures (a bird, which could be a bird of any species, or a completely imaginary bird), bat wing, flying squirrel, a flying lizard, or other real or imaginary flying animal or person), prehistoric or imaginary (dinosaur or dragon, or any real or imaginary animal; a winged reptile, thunderbird, or dragon, or any real or imaginary animal), a four-legged animal (including but not limited to an amphibian, reptile, mammal, dinosaur, human, or other real or imaginary creature with four or more Legs or extremities), letters; numbers; words; a cartoon character, unique or copyrighted cartoon character, a smiley face, jack-o-lantern, moon, sun, or other face or disk-like, oval-like, or face-like representation, leaf or leaves (of any species), an airplane, jet plane, rocket, or other transport device (including automobile, boat, ship or other), a flower (or other plant part), leaves, sun, moon, religious symbols (including cross, shell, Star-of-David, red crescent, lotus blossom, letter, word, or other religious symbol). Also, geometrical forms may be used including by not limited to stars, circles, ovals, lines, stripes, curvilinear designs, spirals, zigzag, or any other abstract or geometrical design with or without color. These changes can be solid or can be intermittent, permitting areas to be translucent or transparent, so that the fluid or contents can still be observed directly.

The invention may be practiced by placing beneficial images an items include syringes, scalpels, needles, bottles, bags, tubing, drapes, chucks, gloves, gowns, headwear, masks, splints, casting material, slings, sheets, bedspreads, drapes, respirators, dialysis machines, compressed gas containers, refuse containers, sharps containers, tubes, canes, walkers, crutches, and any other medical device that could accommodate such changes. Although the designs and ornamentation shown are small relative to the overall size of the device and are repetitive, the designs could be larger and few or single on the medical device.

The beneficial aspects of the present invention have been observed via multiple clinical studies with specific embodiments of this invention reduced to practice Experiments using these design changes on medical devices have demonstrated a predictable and reproducible therapeutic effect—marked reduction of needle-phobia (fear of medical devices), thus, patients are much less frightened, less aversive, and more accepting of medical devices thus ornamented.

EXPERIMENT A: One such experiment took the form of a clinical study of the effects of visually modified infusion and phlebotomy needles on patients. Visually stimulating winged infusion sets were constructed. These included winged needles that looked like real butterflies as well as other fanciful designs. These embodiments in these experiments generally had two symmetrical mirror image decorations each of which replaced either the right or left conventional wings on the needle set, creating a decorated needle with right and left wings that were mirror images of each other. (Specifically, eight different image designs were used and the images used included flowers, smiley faces, animals (two types of fish), 2 types of butterfly, 1 moth, reflective surfaces, stars; the colors used included blue, purple, yellow, metallic, non-metallic, red, green, orange, and black.) Sixty patients were randomized and exposed to conventional infusion/phlebotomy needles and the visually stimulating infusion/phlebotomy needles. Emotional responses to the needles were determined with the Visual Aversion Scale, Analogue Fear Scale, and Analogue Anxiety Scale for each needle design. Results were as follows: The visually stimulating infusion/phlebotomy needles relative to conventional winged catheter sets reduced patient dislike of needles (Visual Aversion Scale: 1.59±2.20 vs. 5.20±3.22, p<0.001), fear of needles (Analogue Fear Scale: 1.62±2.36 vs. 3.86±3.5, p<0.001), and anxiety associated with needles (Analogue Anxiety Scale: 1.89±2.63 vs. 4.02±3.71, p<0.001). This corresponded to a mean 69% reduction in aversion scores, 58% reduction in fear scores, and a 53% reduction in anxiety scores. In terms of individual response, this corresponded to a 95%, 88%, and 81% successful treatment rate, respectively for aversion, fear, and anxiety in individual patients with needle-phobia. When analyzed by individual decorative design, each and every design (8 designs total) had a similar therapeutic effect in reducing needle-phobia—indicating a general therapeutic effect due to decoration of the medical device rather than an effect from an individual decorative design. The different monochrome colors of conventional needles with wings all had similar negative ratings by patients, indicating that monotone color variations do not have a therapeutic effect. Visually stimulating designs on winged catheter sets dramatically reduce patient fear, anxiety, and aversion to needles, most likely by activating brain areas not usually associated with ingrained pain and fear responses.

EXPERIMENT B: A second experiment took the form of a clinical study of the affects of visually modified syringes on patients. Visually stimulating syringes were constructed. These included syringes with dramatic designs on the plunger, so that the visual aspects of the barrel remained optimal. (Specifically, six different plunger designs were used and the images placed on the plungers included raindrops, flowers, musical notes, smiley faces, peace signs, yin-yang, animal (fish), polka-dots, geometric, letters of alphabet, stars, spots; the colors included plain pastel colors and metallic colors, blue, purple, red, yellow, green, black, white, and pink.) Sixty patients were randomized and exposed to conventional syringes (in monochrome colors of green, blue, lime, or violet) and the visually stimulating syringes. Emotional responses to the syringes were determined with the Visual Aversion Scale, Analogue Fear Scale, and Analogue Anxiety Scale for each syringe design. The results were as follows: The visually stimulating syringes relative to conventional syringes reduced patient dislike of syringes (Visual Aversion Scale: 1.21±1.64 vs. 5.88±3.61, p<0.001), fear of syringes (Analogue Fear Scale: 2.19±2.8 vs. 4.68±2.8, p<0.001), and anxiety associated with syringes (Analogue Anxiety Scale: 2.21±2.84 vs. 4.54±3.68, p<0.001). This corresponded to a 79% reduction in aversion scores, 53% reduction in fear scores, and a 51% reduction in anxiety scores. In terms of individual response, this corresponded to a 98%, 87%, and 74% successful treatment rate, respectively for aversion, fear, and anxiety in individual patients with syringe-phobias. When analyzed by individual decorative design, each and every design (6 designs total) had a similar therapeutic effect in reducing syringe-phobia—indicating a general therapeutic effect due to decoration of the medical device rather than an effect from an individual decorative design. Visually stimulating designs on syringes dramatically reduce patient fear, anxiety, and aversion to procedure syringes, most likely by activating brain areas not usually associated with ingrained pain and fear responses.

EXPERIMENT C: Using similar methodology to that used in the infusion and phlebotomy needles study described above, an experiment comparing conventional intravenous infusion (IV) bags with IV bags modified to be visually stimulating with fish, dolphins, flowers, or smiley faces. The following results were obtained:

| | Visually Stimulating | Conventional | |
|---|---|---|---|
| Ten Subjects | IV Bag | IV Bag | P value |
| Aversion Score | 0.82 ± 1.2 | 4.95 ± 2.42 | 0.0003 |
| Fear Score | 0.62 ± 1.35 | 1.5 ± 2.69 | 0.24 |
| Anxiety Score | 0.75 ± 1.69 | 2.75 ± 3.43 | 0.05 |

Thus, IV bags according to the present invention decreased aversion (dislike) of IV bags by 83%, fear by 59%, and anxiety by 72%. This corresponded to a 90% response rate (treatment success). in individual patients with originally negative feelings toward IV therapy.

EXPERIMENT D: Using similar methodology to that used in the infusion and phlebotomy needles study described above, an experiment comparing conventional scalpels with scalpels modified to have visually stimulating handles. (stars or musical notes) The following results were obtained:

| | Visually Stimulating | Conventional | |
|---|---|---|---|
| Ten Subjects | Scalpel | Scalpel | P value |
| Aversion Score | 3.62 ± 3.25 | 7.10 ± 3.02 | 0.0004 |
| Fear Score | 4.50 ± 3.45 | 7.0 ± 3.29 | 0.003 |
| Anxiety Score | 4.25 ± 3.69 | 6.70 ± 1.12 | 0.005 |

Thus, scalpels according to the present invention decreased patient aversion (dislike) of scalpels by 49%, fear of scalpels by 36%, and anxiety of scalpels by 37%. This corresponded to a 90% response rate (treatment success). in individual patients with extreme fear of scalpels.

The empirical data indicate that decorating a medical device is a reproducible method with a beneficial, and predictable therapeutic function of decreasing a patient's fear, anxiety, aversion, and pain associated with a medical procedure and the associated medical device. This effect is not specific to the individual decorative design, but rather is a general phenomenon related to decorations on a medical device used in procedures. This technique is without precedent because it is functionally and temporally contrasted with the decoration of wound dressings. When a wound dressing has been decorated, the documented benefits of a visually stimulating medical device cannot accrue because a wound dressing is applied after the painful procedure has already been performed. The use of a visually stimulating medical device prior to and during the painful procedure decreases a patient's fear, anxiety, aversion, and pain at a much more useful stage of treatment, when the adverse emotions and pain are actually occurring. The high response rates of this new patient-oriented approach to medical device design will be of considerable benefit to patients when methodically applied to medical devices and consumables typically used in procedures and therapies.

The present invention is practiced by applying decorations or designs to medical devices and medical consumables used in procedures in a calculated way in order to visually activate parts of the patient's brain sensory centers not usually associated with the emotions of fear, anxiety, dislike, and pain. By doing this, the patient's emotions and sensations of fear, anxiety, dislike, and pain experiences during a procedure are actually decreased. The data presented demonstrate in the cases of winged needles, syringes, IV bags, and scalpels the decorations dramatically decrease a patient's fear, dislike, and anxiety associated with these medical devices. The response rates are very high, in the range of 90%, which is excellent for any therapeutic intervention in medicine. One mode of practicing the invention is for these decorations to be applied to the medical devices using methods known to those skilled in the art during the manufacturing process, so that the decorated devices could be taken out of the packaging and used immediately without modification by the healthcare providers.

Humans typically decorate things they like, not that they dislike—thus, the invention here is completely counterintuitive, which explains both its novelty and effectiveness Decorations are usually considered a design patent or a trademarked pattern, but the data presented here demonstrate that a wide variety of decorations and forms and colors, both abstract and representative, each have the similar effect of reducing a patient's fear, dislike, and anxiety of medical devices used in procedures. Uniform or solid color, that is, blue, green, etc already exist in medical devices and do not appreciably change a patient's perception of the devices—our data (see EXPERIMENT A) demonstrate this. Simply changing the color of a medical device from black to green, or blue to purple does not decrease a patient's adverse reactions to medical devices. Rather, it is the decoration itself that is necessary for the beneficial effect to be noted. The decorations apparently stimulate areas of the patient's brain that diminish the sensations of dislike, fear, anxiety, and pain usually provoked by standard, undecorated devices. Thus, these decorated devices have a powerful and reproducible therapeutic effect.

Referring to FIG. 27, a medical tube according to the prior art without an image imprint is illustrated. This is an example of the existing medical tubing that are formed so as to have an appearance that tends to promote confusion.

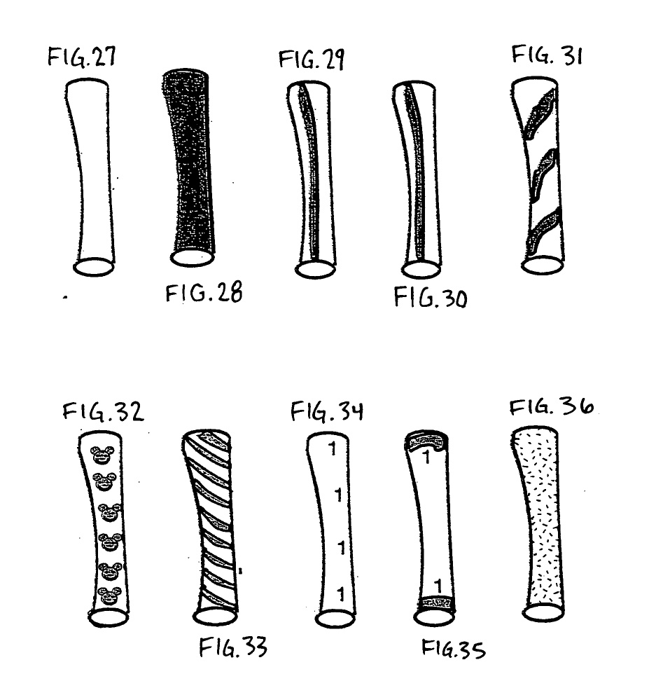

Referring to FIGS. 28-36, examples are illustrated of tubing having imprinted images that provide not only the benefits noted above but also coding for intravenous and other forms of tubing. Referring to FIG. 28, medical tubing imprinted with a coded coloration across substantially the entire tube surface is illustrated. Referring to FIGS. 29-31 and 33, medical tubing imprinted with a coded coloration in striped, linear, and spiral patterns is illustrated. Geometrical shapes may also be used. Referring to FIG. 32, medical tubing imprinted with a repetitive pattern based on a literary character motif is illustrated. Any of various artistic representations, cartoons, or soothing graphics may be used. Referring to FIG. 34, medical tubing imprinted with coded symbols is illustrated. Referring to FIG. 35, medical tubing imprinted with a combination of coded coloration and coded symbols is illustrated. Referring to FIG. 36, medical tubing with a modified surface texture is illustrated.

This type of coding need not be on the tubing or catheter or other medical device itself. Rather, the coding is alternatively placed on the fittings of the tubing, catheter or medical device or could be on both the fittings and the tubing or medical device. In this way, analogous to the established coding for electrical wiring, medical tubing and other medical devices can be differentiated from one another, preventing misadministration or measurement of a particular medication or other measure from a tubing. These changes are solid or continuous or repetitive or intermittent, permitting areas to be translucent or transparent, so that the fluid or contents can still be observed directly. These changes also permit two or more components to be correctly assembled by having identical or sequential (as in the case of letters or numbers) coding on those components or ends to be assembled together.

The present invention has been described in terms of various exemplary embodiments. Persons of ordinary skill in the art will understand that various improvements, changes, and modifications may be made to the illustrated embodiments without departing from the scope of the present invention.

1. A medical device for use in the vicinity of a patient and having an exterior surface, the medical device comprising:
an image imprinted on the exterior surface of the medical device adapted to provide a calming affect for the patient.

2. The medical device of claim 1, wherein the medical device comprises a syringe.

3. The medical device of claim 1, wherein the medical device comprises a scalpel.

4. The medical device of claim 1, wherein the medical device comprises an intravenous infusion bag.

5. The medical device of claim 1, wherein the medical device comprises a needle or needle cap.

6. The medical device of claim 1, wherein the medical device comprises a surgical mask.

7. The medical device of claim 1, wherein the medical device comprises a blood drawing device.

8. A syringe that provides a calming affect for a patient, the syringe comprising:
a barrel; and
a plunger disposed inside the barrel;
wherein at least the plunger is imprinted with images that provide a calming affect for the patient.

9. A scalpel that provides a calming affect for a patient, the scalpel comprising:
a blade; and
a handle connected to the blade;
wherein at least the handle is imprinted with images that provide a calming affect for the patient.

10. An intravenous infusion bag that provides a calming affect for a patient, the bag comprising:
a bag body; and
a fluid conduit formed at one end of the bag body;
wherein at least the bag body is imprinted with images that provide a calming affect for the patient.

11. A needle cap that provides a calming affect for a patient, wherein at least a portion of the needle cap is imprinted with images that provide a calming affect for the patient.

12. A surgical mask that provides a calming affect for a patient, the surgical mask comprising:
a mask body; and
a mask retention members connected to the mask body;
wherein at least the mask body is imprinted with images that provide a calming affect for the patient.

13. A blood drawing device that provides a calming affect for a patient, wherein at least a portion of the blood drawing device is imprinted with images that provide a calming affect for the patient.

14. A fluid conduit for medical use, wherein the fluid conduit is imprinted with a color and visual pattern indicative of a particular medical use for the fluid conduit.

15. A fluid conduit for medical use, wherein the fluid conduit is imprinted with a palpable texture indicative of a particular medical use for the fluid conduit.

16. A method for providing a calming affect for a patient, the method comprising:
imprinting an image on a medical device; and
deploying the medical device so as to place the image within view of the patient;
wherein viewing of the image by the patient provides a calming affect for the patient.
